(19) United States
(12) Patent Application Publication
(10) Pub. No.: US 2025/0354215 A1
(43) Pub. Date: Nov. 20, 2025

(54) METHOD FOR DETECTING HIGH-RISK NASOPHARYNGEAL CANCER

(71) Applicants: National University Of Singapore, Singapore (SG); National University Hospital (Singapore) Pte Ltd, Singapore (SG)

(72) Inventors: Kai Xun Joshua TAY, Singapore (SG); Kwok Seng LOH, Singapore (SG); Wei Keat TEO, Singapore (SG); Woei Shyang LOH, Singapore (SG); Luvita SURYANI, Singapore (SG); Bingcheng WU, Singapore (SG)

(73) Assignees: National University Of Singapore, Singapore (SG); National University Hospital (Singapore) Pte Ltd, Singapore (SG)

(21) Appl. No.: 18/854,448

(22) PCT Filed: Apr. 6, 2023
(86) PCT No.: PCT/SG2023/050239

§ 371 Date: Oct. 4, 2024

(30) Foreign Application Priority Data
Apr. 7, 2022 (SG) 10202203606T

(51) Int. Cl. C12Q 1/6886; C40B 40/06

(52) U.S. Cl. CPC C12Q 1/6886; C40B 40/06; C12Q 2600/112; C12Q 2600/118; C12Q 2600/158

(57) ABSTRACT

The invention relates generally to the field of oncology. Provided herein is a method for predicting the likelihood of recurrence or prognosis of a cancer in a subject, comprising comparing the level of expression of one or more biomarkers selected from the group consisting of Fibroblast Growth Factor 2 (FGF2), Complement Component 3 (C3), and GTPase, IMAP Family Member 7 (GIMAP7) in a sample obtained from the subject to a reference. In particular, the cancer is nasopharyngeal cancer. Also provided is a composition or solid support comprising a plurality of DNA/RNA complexes, wherein each DNA/mRNA complex in the plurality comprises a biomarker and a first and second DNA probe hybridized to the biomarker, wherein the plurality of DNA/mRNA complexes comprise one or more biomarkers selected from the group consisting of FGF2, C3, and GIMAP7.

### FIELD OF INVENTION

The invention relates to generally to the field of oncology. Provided herein is a method for detecting for detecting and classifying nasopharyngeal cancer.

### BACKGROUND

Nasopharyngeal cancer (NPC) is a cancer that occurs in the nasopharynx. While NPC is rare in the United States, it occurs frequently in other parts of the world, such as in Southern China and Southeast Asia. It is the second most common cancer in middle-aged men in Singapore. Other risk factors of NPC include being exposed to the Epstein-Barr virus or excessive alcohol consumption. The cancer is difficult to detect early and has a high rate of disease recurrence of about 30% to 40%. Furthermore, treatment options are limited and are often associated with significant morbidity. There is currently no molecular method to accurately identify patients who are at high risk of recurrence.

It would be desirable to overcome or ameliorate at least one of the above-described problems, or at least to provide a useful alternative.

### SUMMARY

Disclosed herein is a method of predicting the likelihood of recurrence of a cancer in a subject, the method comprises comparing the level of expression of one or more biomarkers selected from the group consisting of Fibroblast Growth Factor 2 (FGF2), Complement Component 3 (C3) and GTPase, IMAP Family Member 7 (GIMAP7) in a sample obtained from the subject to a reference, wherein a change in level of expression in the one or more biomarkers, or a value derived therefrom, as compared to the reference predicts the likelihood of recurrence of the cancer in the subject.

Disclosed herein is a method of determining the prognosis of a cancer in a subject, the method comprises comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference indicates that the subject is likely to have a high risk cancer or a low risk cancer.

Disclosed herein is a method of treating cancer in a subject, the method comprises a) comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference indicates that the subject is likely to have a high risk cancer; and b) administering an anti-cancer agent and/or radiotherapy to a subject found likely to have a high risk cancer.

In one embodiment, the subject found likely to have a high risk cancer is to be followed-up more closely. The subject may be administered further treatment to reduce the risk of recurrence of cancer. For example, the subject may be administered a second-line anti-cancer agent. The subject may also be given a targeted therapy against FGF2 and FGFR. The subject may also be given an immunotherapy or an anti-vascular endothelial growth factor therapy.

Also disclosed herein is a method of stratifying a subject into one who is suffering from a high risk or low risk cancer, the method comprises comparing the level of expression of one or more biomarkers selected from the group consisting of FGF2, C3 and GIMAP7 in a sample obtained from the subject to a reference, wherein a change in level of expression in the one or more biomarkers, or the value derived therefrom, as compared to the reference stratifies the subject into one who is suffering from a high risk or low risk cancer.

Disclosed herein is a composition or solid support comprising a plurality of DNA/mRNA complexes, wherein each DNA/mRNA complex in the plurality comprises a biomarker and a first and second DNA probe hybridized to the biomarker, wherein:

the first probe is a capture probe; the second probe is a reporter probe; the biomarker is a mRNA transcript; and the plurality of DNA/mRNA complexes comprise one or more biomarkers selected from the group consisting of FGF2, C3 and GIMAP7.

### BRIEF DESCRIPTION OF DRAWINGS

Embodiments of the present invention are hereafter described, by way of non-limiting example only, with reference to the accompanying drawings in which:

FIG. 1(a) Heat map of differentially expressed genes in the micro-dissected tumor epithelial compartment in a discovery cohort comparing between primary NPC tumors that eventually recurred and tumors that did not recur (n=34 gene expression libraries). (b) Unsupervised hierarchical clustering showed that the three gene signature accurately classified tumors that recurred and tumors that did not recur in the discovery cohort.

FIG. 2(a) Unsupervised hierarchical clustering showed that the three gene signature defined tumor clusters based on their risk profiles for developing recurrent disease in a first validation cohort. (b) A risk score derived from the three gene signature showed that tumors which recurred had higher risk scores compared to tumors that did not recur. (c) Survival analysis based on Kaplan-Meier curve plots show that patients with higher risk score tumors have poorer disease-free survival compared to patients with lower risk score tumors.

FIG. 3(a) Unsupervised hierarchical clustering showed that the three gene signature defined tumor clusters based on their risk profiles for developing recurrent disease a second validation cohort. (b) A risk score derived from the three gene signature showed that tumors which recurred had higher risk scores compared to tumors that did not recur. (c) Survival analysis based on Kaplan-Meier curve plots show that patients with higher risk score tumors have poorer disease-free survival compared to patients with lower risk score tumors.

FIG. 4(a) Representative images of cell migration assay (scratch assay) showing increased migration of C666-1 cells with media containing FGF2 at 5 ng/ml compared to control. (b) Quantification of cells migrating to the wounded area of the same cell migration assay. (c) Cell invasion assay showing increased invasion of C666-1 cells when incubated with media containing FGF2 compared to control.

### DETAILED DESCRIPTION

The present specification teaches a method of predicting the likelihood of recurrence of a cancer in a subject.

Disclosed herein is a method of predicting the likelihood of recurrence of a cancer in a subject, the method comprises comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference predicts the likelihood of recurrence of the cancer in the subject.

Without being bound by theory, the inventors have developed a microdissected gene expression classifier that performs robustly across different methodologies to quantify gene expression (bulk or microdissected). The genes identified here are biologically relevant. FGF2 is a potentially actionable protein, as clinical trials have been performed in other solid-organ cancers targeting this pathway. FGFR inhibitors have also been approved for clinical use in other cancers. C3 is a critical member of complement pathway involved in innate immunity, while GIMAP GTPases are also key immune mediators which can be potentially targeted.

The term “biomarker” refers to a measurable characteristic that reflects the presence or nature (e.g., severity) of a physiological and/or pathophysiological state, including an indicator of risk of developing a particular physiological or pathophysiological state, such as cancer. Biomarkers may be present in a sample obtained from a subject before the onset of a physiological or pathophysiological state, including a symptom, thereof. Thus, the presence of the biomarker in a sample obtained from the subject is likely to be indicative of an increased risk that the subject will develop the physiological or pathophysiological state or symptom thereof. Alternatively, or in addition, the biomarker may be normally expressed in an individual, but its expression may change (i.e., it is increased (upregulated; over-expressed) or decreased (downregulated; under-expressed) before the onset of a physiological or pathophysiological state, including a symptom thereof. Thus, a change in the level of expression of the biomarker is likely to be indicative of an increased risk that the subject will develop the physiological or pathophysiological state or symptom thereof.

Biomarkers include, for example, gene expression products, including mRNA transcripts and peptides or proteins expressed from the gene, metabolites etc. It is understood that reference to a gene as a biomarker (also sometimes referred to herein as a biomarker gene) means that the product of the gene is the biomarker, i.e. the mRNA transcript and/or the protein expressed from the biomarker gene is the biomarker. Thus, for example, reference to FGF2 as a biomarker refers to the mRNA transcript from the FGF2 gene and/or the protein expressed from the FGF2 gene. As herein described, reference to the expression of a biomarker includes the concentration, level or activity of the biomarker, such as the concentration or level of a mRNA transcript or the concentration, or the concentration, level or activity of a protein. For example, where the biomarker is an enzyme, its expression may be determined or measured by the level of activity of the enzyme on a known substrate.

The term “expression” with respect to a gene refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The term “expression product” or “gene expression product” or “gene product” are used herein to refer to the RNA transcription products (transcripts) of a gene, including mRNA, and the polypeptide translation products of such RNA transcripts. An expression product can be, for example, an unspliced RNA, an mRNA, a splice variant mRNA, a microRNA, a fragmented RNA, a polypeptide, a post-translationally modified polypeptide, a splice variant polypeptide, etc.

The term “gene” as used herein refers to any and all discrete coding regions of the cell's genome, as well as associated non-coding and regulatory regions. The term “gene” is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

As used herein the terms “level” and “amount” are used interchangeably to refer to a quantitative amount (e.g., weight or moles or number), a semi-quantitative amount, a relative amount (e.g., weight % or mole % within class or a ratio), a concentration, and the like. Thus, in reference to the amount or level of a biomarker, the terms encompasses absolute or relative amounts or concentrations of biomarkers in a sample, including ratios of levels of biomarkers, and odds ratios of levels or ratios of odds ratios. Levels or amounts may also be reflective of an individual subject or of cohorts of subjects, the latter being expressed, for example, as mean or medium levels.

The term “nucleic acid” or “polynucleotide” as used herein designates mRNA, RNA, CRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA or RNA. “Protein,” “polypeptide” and “peptide” are also used interchangeably herein to refer to a polymer of amino acid residues and to variants and synthetic analogues of the same.

As used herein, “obtained” is meant to come into possession. For example, reference to obtaining a biomarker profile can include coming into possession of an already generated profile, such as by accessing the profile from a computer or database, as well as generating the profile by evaluating the relevant biomarkers. In another example, obtaining a sample, such as a biological sample, can include coming into the possession of a sample that has already been taken from a subject, as well as actively taking a sample from a subject.

In one embodiment, the method comprises detecting the level of the one or more biomarkers. The level of the one or more biomarkers may be detected by techniques well known in the art (such as RNA sequencing). In one embodiment, the method comprises or consist of comparing the levels of FGF2, C3 or GIMAP7. In one embodiment, the method comprises or consist of comparing the levels of FGF2 and C3. In one embodiment, the method comprises or consist of comparing the levels of FGF2 and GIMAP7. In one embodiment, the method comprises or consist of comparing the levels of C3 and GIMAP7. In one embodiment, the method comprises or consists of comparing the levels of FGF2, C3 and GIMAP7.

Disclosed herein is a method of determining the prognosis of a cancer in a subject, the method comprises comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference indicates that the subject is likely to have a high risk cancer or a low risk cancer.

The term “prognosis” as referred to herein refers to a prediction of the probable course and outcome of a clinical condition or disease. A prognosis of a patient is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, glioma, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In one embodiment, the cancer is nasopharyngeal cancer (NPC). In one embodiment, the cancer is a metastatic cancer. In one embodiment, the cancer is a metastatic nasopharyngeal cancer (NPC).

The terms “subject”, “individual” and “patient”-which are used interchangeably herein, are intended to refer to any subject, preferably a mammalian subject, and more preferably still a human subject. Mammalian subjects include humans, domestic animals, farm animals, sports animals, and zoo animals including, e.g., humans, non-human primates, dogs, cats, mice, rats, guinea pigs, and the like. In some embodiments, the subject has, or is suspected of having, a nasopharyngeal cancer (NPC).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

A sample can be a biological sample which refers to the fact that it is derived or obtained from a living organism. The organism can be in vivo (e.g. a whole organism) or can be in vitro (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is ascites. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. In one embodiment, the sample is a tissue sample. The tissue sample may be a fresh tissue, frozen tissue or paraffin-embedded formalin-fixed (FFPE) tissue sample. In one embodiment, the tissue sample is obtained by microdissection. In one embodiment, the sample is obtained by laser-capture microdissection.

The biological sample may be processed and analyzed for the purpose of evaluating the biomarkers almost immediately following collection (i.e., as a fresh sample), or it may be stored for subsequent analysis. If storage of the biological sample is desired or required, it would be understood by persons skilled in the art that it should ideally be stored under conditions that preserve the integrity of the biomarker of interest within the sample (e.g., at −80° C.).

Evaluation of a biomarker may comprise evaluation of the level of mRNA expressed from the recited gene and/or the level of protein expressed from the recited gene. Methods of measuring expression products such as transcripts and proteins are well known to persons skilled in the art, with some illustrative examples described below.

Nucleic acid-based assays are well known in the art and include low-throughput and high throughput assays. In illustrative nucleic acid-based assays, nucleic acid is isolated from cells contained in a biological sample according to standard methodologies (Sambrook, et al., 1989, supra; and Ausubel et al., 1994, supra). The nucleic acid is typically fractionated (e.g., poly A+RNA) or whole cell RNA. Where RNA is used as the subject of detection, it may be desired to convert the RNA to a complementary DNA. In some embodiments, the nucleic acid is amplified by a template-dependent nucleic acid amplification technique. A number of template dependent processes are available to amplify the biomarker sequences present in a given template sample. An exemplary nucleic acid amplification technique is the polymerase chain reaction (referred to as PCR), which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the biomarker sequence. An excess of deoxynucleotide triphosphates are added to a reaction mixture along with a DNA polymerase, e.g., Taq polymerase. If a cognate biomarker sequence is present in a sample, the primers will bind to the biomarker and the polymerase will cause the primers to be extended along the biomarker sequence by adding on nucleotides. By raising and lowering the temperature of the reaction mixture, the extended primers will dissociate from the biomarker to form reaction products, excess primers will bind to the biomarker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art.

In certain embodiments, the template-dependent amplification involves quantification of transcripts in real-time. For example, RNA or DNA may be quantified using the Real-Time PCR technique (Higuchi, 1992, et al., Biotechnology 10:413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA. In specific embodiments, multiplexed, tandem PCR (MT-PCR) is employed, which uses a two-step process for gene expression profiling from small quantities of RNA or DNA, as described for example in US Pat. Appl. Pub. No. 20070190540. In the first step, RNA is converted into cDNA and amplified using multiplexed gene specific primers. In the second step each individual gene is quantitated by real time PCR.

In certain embodiments, target nucleic acids are quantified using blotting techniques, which are well known to those of skill in the art. Southern blotting involves the use of DNA as a target, whereas Northern blotting involves the use of RNA as a target. Each provides different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labelled) under conditions that promote denaturation and rehybridisation. Because the probe is designed to base pair with the target, the probe will bind a portion of the target sequence under renaturing conditions. Unbound probe is then removed, and detection is accomplished as described above. Following detection/quantification, one may compare the results seen in a given subject with a control reaction or a statistically significant reference group or population of control subjects as defined herein.

Also contemplated are biochip-based technologies such as those described by Hacia et al. (1996, Nature Genetics 14:441-447) and Shoemaker et al. (1996, Nature Genetics 14:450-456). Briefly, these techniques involve quantitative methods for analyzing large numbers of genes rapidly and accurately. By tagging genes with oligonucleotides or using fixed probe arrays, one can employ biochip technology to segregate target molecules as high-density arrays and screen these molecules on the basis of hybridization. See also Pease et al. (1994, Proc. Natl. Acad. Sci. U.S.A. 91:5022-5026); Fodor et al. (1991, Science 251:767-773). Briefly, nucleic acid probes to biomarker polynucleotides are made and attached to biochips to be used in screening and diagnostic methods, as outlined herein. The nucleic acid probes attached to the biochip are designed to be substantially complementary to specific expressed biomarker nucleic acids, i.e., the target sequence (either the target sequence of the sample or to other probe sequences, for example in sandwich assays), such that hybridization of the target sequence and the probes of the present invention occur. This complementarity need not be perfect; there may be any number of base pair mismatches, which will interfere with hybridization between the target sequence and the nucleic acid probes of the present invention. However, if the number of mismatches is so great that no hybridization can occur under even the least stringent of hybridization conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e. have some sequence in common), or separate.

In an illustrative biochip analysis, oligonucleotide probes on the biochip are exposed to or contacted with a nucleic acid sample suspected of containing one or more biomarker polynucleotides under conditions favouring specific hybridization. Sample extracts of DNA or RNA, either single or double-stranded, may be prepared from fluid suspensions of biological materials, or by grinding biological materials, or following a cell lysis step which includes, but is not limited to, lysis effected by treatment with SDS (or other detergents), osmotic shock, guanidinium isothiocyanate and lysozyme. Suitable DNA, which may be used in the method of the invention, includes cDNA. Such DNA may be prepared by any one of a number of commonly used protocols as for example described in Ausubel, et al., 1994, supra, and Sambrook, et al., et al., 1989, supra.

Methods for assessing mRNA levels that do not require conversion of the mRNA to cDNA are also known in the art and are suitable for the operation of the present invention. In a particular example, digital molecular barcoding technology is used to measure mRNA levels. In such techniques, including, for example, NanostringnCounter™, color-coded molecular barcodes are utilized in a multiplex assay For example, in such a method each color-coded barcode is attached to a target-specific reporter probe, for example about 50 bases to about 100 bases or any number between 50 and 100 bases in length that hybridizes to a gene of interest. Two probes are used to hybridize to mRNA transcripts of interest: the reporter probe that carries the color signal and a capture probe that allows the probe-target complex to be immobilized on to a solid support for data collection. The probe-target complexes can be immobilized on a substrate for data collection, for example an nCounter™Cartridge and analyzed for example in a Digital Analyzer such that color codes are counted and tabulated for each target molecule.

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. 1994, supra and Sambrook, et al. 1989, supra).

cDNA may be fragmented, for example, by sonication or by treatment with restriction endonucleases. Suitably, cDNA is fragmented such that resultant DNA fragments are of a length greater than the length of the immobilized oligonucleotide probe(s) but small enough to allow rapid access thereto under suitable hybridization conditions. Alternatively, fragments of cDNA may be selected and amplified using a suitable nucleotide amplification technique, as described for example above, involving appropriate random or specific primers.

The target biomarker polynucleotides (e.g. mRNA or cDNA) or a probe that hybridizes to the target polynucleotide is typically detectably labelled so that the hybridization can be detected. Detectable labels include, for example, chromogens, catalysts, enzymes, fluorochromes, chemiluminescent molecules, bioluminescent molecules, lanthanide ions (e.g., Eu34), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like. Illustrative labels of this type include large colloids, for example, metal colloids such as those from gold, selenium, silver, tin and titanium oxide. In some embodiments, in which an enzyme is used as a direct visual label, biotinylated bases are incorporated into a target polynucleotide.

The hybrid-forming step can be performed under suitable conditions for hybridizing oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridization. The preferred conditions will therefore depend upon the particular application. Such empirical conditions, however, can be routinely determined without undue experimentation.

After the hybrid-forming step, typically, the probes are washed to remove any unbound nucleic acid with a hybridization buffer. This washing step leaves only bound target polynucleotides. The probes are then examined to identify which probes have hybridized to a target polynucleotide.

The hybridization reactions are then assessed to detect the target polynucleotide/probe complexes. Depending on the nature of the reporter molecule associated with a target polynucleotide or probe, a signal may be instrumentally detected by irradiating a fluorescent label with light and detecting fluorescence in a fluorimeter; by providing for an enzyme system to produce a dye which could be detected using a spectrophotometer; or detection of a dye particle or a coloured colloidal metallic or non-metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focused beam or laser light. In such a case, a charge couple device (CCD) or a photocell can be used to scan for emission of light from a probe: target polynucleotide hybrid from each location in the micro-array and record the data directly in a digital computer. In some cases, electronic detection of the signal may not be necessary. For example, with enzymatically generated colour spots associated with nucleic acid array format, visual examination of the array will allow interpretation of the pattern on the array. In the case of a nucleic acid array, the detection means is suitably interfaced with pattern recognition software to convert the pattern of signals from the array into a plain language genetic profile. In certain embodiments, oligonucleotide probes specific for different biomarker polynucleotides are in the form of a nucleic acid array and detection of a signal generated from a reporter molecule on the array is performed using a ‘chip reader’. A detection system that can be used by a ‘chip reader’ is described for example by Pirrung et al (U.S. Pat. No. 5,143,854). The chip reader will typically also incorporate some signal processing to determine whether the signal at a particular array position or feature is a true positive or maybe a spurious signal. Exemplary chip readers are described for example by Fodor et al (U.S. Pat. No. 5,925,525). Alternatively, when the array is made using a mixture of individually addressable kinds of labelled microbeads, the reaction may be detected using flow cytometry.

In other embodiments, the level of protein expressed from a gene is evaluated, such as using protein-based assays known in the art. Antibody-based techniques may also be employed to determine the level of a biomarker in a sample, non-limiting examples of which include immunoassays, such as the enzyme-linked immunosorbent assay (ELISA) and the radioimmunoassay (RIA).

In specific embodiments, protein-capture arrays that permit simultaneous detection and/or quantification of a large number of proteins are employed. For example, low-density protein arrays on filter membranes, such as the universal protein array system (Ge, 2000 Nucleic Acids Res. 28 (2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, BioRad and Sigma). Various methods for the preparation of antibody arrays have been reported (see, e.g., Lopez et al., 2003 J. Chromatogr. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognize at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors and cell-surface antigens.

Individual spatially distinct protein-capture agents are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include colour coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtitre plate or in separate test tubes.

In an illustrative example, a protein sample, which is optionally fragmented to form peptide fragments (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second protein in the sample is already known or known to be invariant.

In another illustrative example of a protein-capture array is Luminex-based multiplex assay, which is a bead-based multiplexing assay, where beads are internally dyed with fluorescent dyes to produce a specific spectral address. Biomolecules (such as an oligo or antibody) can be conjugated to the surface of beads to capture analytes of interest.

Flow cytometric or other suitable imaging technologies known to persons skilled in the art can then be used for characterization of the beads, as well as for detection of analyte presence. The Luminex technology enables are large number of proteins, genes or other gene expression products (e.g., 100 or more, 200 or more, 300 or more, 400 or more) to be detected using very small sample volume (e.g., in a 96 or 384-well plate). In some embodiments, the protein-capture array is Bio-Plex Luminex-100 Station (Bio-Rad) as described previously.

For analyzing differential expression of proteins between two cells or cell populations, a protein sample of a first cell or population of cells is delivered to the array under conditions suitable for protein binding. In an analogous manner, a protein sample of a second cell or population of cells to a second array is delivered to a second array that is identical to the first array. Both arrays are then washed to remove unbound or non-specifically bound components of the sample from the arrays. In a final step, the amounts of protein remaining bound to the features of the first array are compared to the amounts of protein remaining bound to the corresponding features of the second array. To determine the differential protein expression pattern of the two cells or populations of cells, the amount of protein bound to individual features of the first array is subtracted from the amount of protein bound to the corresponding features of the second array.

In other example, when a biomarker protein is an enzyme, the protein can be quantified based upon its catalytic activity or based upon the number of molecules of the protein contained in a sample.

In some embodiments, the level of a biomarker is normalized against a housekeeping biomarker (i.e. is relative to the housekeeping biomarker), or is expressed as a ratio between the level of the biomarker a and the level of the housekeeping biomarker. Housekeeping biomarkers are biomarkers or a group of biomarkers (e.g., polynucleotides and/or polypeptides), which are typically found at a constant level in the cell type(s) being analyzed and across the conditions being assessed. In some embodiments, the housekeeping biomarker is a housekeeping gene, i.e. a gene or group of genes which encode proteins whose activities are essential for the maintenance of cell function and which are typically found at a constant level in the cell type(s) being analyzed and across the conditions being assessed.

In one embodiment, the method comprises comparing the level of expression of the mRNA or protein of the one or more biomarkers.

The term “reference” may refer to a sample from a healthy individual (such as one who does not have a glioma) or may refer to a non-cancerous sample. Alternatively, the “reference” may refer to a sample from an individual with cancer or may refer to a cancer (such as an NPC) sample. It may also refer to a pre-determined value.

A change in level of expression in the one or more biomarkers, or a value derived therefrom, as compared to the reference may refer to a change in the level of expression in the one or more biomarkers or a change in a value derived therefrom the level of expression in the one or more biomarkers. The change may be an increase or decrease in the level of expression in the one or more biomarkers. It may also be an increase or decrease in the value derived therefrom the level of expression in the one or more biomarkers.

As used herein, the term “increase” or “increased” in the level of the one or more biomarkers, or a value derived therefrom, refers to a statistically significant and measurable increase as compared to a reference. The increase may refer to an increase of at least about 10%, or an increase of at least about 20%, or an increase of at least about 30%, or an increase of at least about 40%, or an increase of at least about 50% or more.

In one embodiment, an increased level of the one or more biomarkers, or a value derived therefrom, as compared to a reference predicts the likelihood of recurrence of the cancer in the subject. The increase may be an increase of 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a reference.

As used herein, the term “decrease” or “decreased” in the level of the one or more biomarkers, or a value derived therefrom, refers to a statistically significant and measurable decrease as compared to a reference. The decrease may refer to a decrease of at least about 10%, or a decrease of at least about 20%, or a decrease of at least about 30%, or a decrease of at least about 40%, or a decrease of at least about 50% or more.

In one embodiment, the value derived from the level of the one or more biomarkers is a probability. The probability may be derived from a logistic regression model, and wherein the level of the one or more biomarkers is the predictor of the logistic regression model. The value may be compared to a reference, which may, for example, be a cutoff designated at 0.5.

In one embodiment, the value derived therefrom is a cancer recurrence risk score obtained by processing the level of expression of one or more biomarkers selected from the group consisting of FGF2, C3 and GIMAP7 using a risk score model.

In one embodiment, the risk score model is generated by regression on a dataset comprising gene expression data and clinical outcome of a cohort of cancer patients.

The term “recurrence” as used herein may refer to a cancer that has recurred (come back), usually after a period of time during which the cancer could not be detected. The cancer may be called a recurrent cancer. The recurrent cancer may come back to the same place as the original (primary) tumor or to another place in the body. The recurrence may be considered a “local recurrence” when the cancer is in the same place as the original cancer or very close to it. The recurrence may be a “regional recurrence” when the tumor has grown into lymph nodes or tissues near the original cancer. The recurrence may be called a distant recurrence when the cancer has spread to organs or tissues far from the original cancer. When the cancer spreads to a distant place in the body, the recurrent cancer may be called metastasis or metastatic cancer.

The term “likelihood of recurrence” may refer to how likely it is for a cancer to recur in a subject. An increase in the level of the one or more biomarkers, or a value derived therefrom, as compared to a reference may indicate a 10%, 20%, 30%, 40%, 50%, 60%, 70%, 80%, 90% or 99% or more likelihood of recurrence in the subject.

It would be apparent to persons skilled in the art that the predicted likelihood of cancer recurrence in a subject will vary, for example, from being at low (including negligible) or decreased likelihood to being at high or increased likelihood of recurrence. A subject with a low or decreased likelihood of recurrence means that the cancer is less likely to recur in the subject as compared to a subject with a high or increased likelihood of recurrence. Conversely, a subject with a high or increased likelihood of recurrence means that the cancer is more likely to recur in the subject as compared to a subject with a low or decreased likelihood of recurrence.

Likelihood is suitably based on mathematical modeling. An increased likelihood, for example, may be relative or absolute and may be expressed qualitatively or quantitatively. For instance, an increased risk may be expressed as simply determining the subject's level of a given biomarker at one or more time points and placing the test subject in an “increased risk” category, based upon a reference (e.g. a reference biomarker) as determined, for example, from previous population studies at the same time points. Alternatively, a numerical expression of the test subject's increased risk may be determined based upon biomarker level analysis.

In some embodiments, likelihood is assessed by comparing the level or abundance of at least one biomarker to one or more preselected level, also referred to herein as a threshold or reference levels. Thresholds may be selected that provide an acceptable ability to predict risk, treatment success, etc. In illustrative examples, receiver operating characteristic (ROC) curves are calculated by plotting the value of a variable versus its relative frequency in two populations in which a first population is considered at risk of cancer recurrence and a second population that is not considered to be at risk, or have a low risk, of cancer recurrence (called arbitrarily, for example, “healthy controls”).

In some embodiments, the subject is considered at risk of cancer recurrence where at least one biomarker in the sample biomarker profile for the subject is upregulated or downregulated as compared to the corresponding biomarker in a healthy subject, as described herein.

For any particular biomarker, a distribution of biomarker levels for subjects who are at risk or not at risk of cancer recurrence may overlap. Under such conditions, a test may not absolutely distinguish a subject who is at risk of cancer recurrence from a subject who is not at risk of cancer recurrence with absolute (i.e., 100%) accuracy, and the area of overlap indicates where the test cannot distinguish the two subjects. A threshold can be selected, above which (or below which, depending on how a biomarker changes with risk) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143:29-36 (1982)).

In some embodiments, a positive likelihood ratio, negative likelihood ratio, odds ratio, and/or AUC or receiver operating characteristic (ROC) values are used as a measure of a method's ability to predict risk of cancer recurrence. As used herein, the term “likelihood ratio” is the probability that a given test result would be observed in a subject with a likelihood of such risk, divided by the probability that that same result would be observed in a subject without a likelihood of such risk. Thus, a positive likelihood ratio is the probability of a positive result observed in subjects with the specified risk divided by the probability of a positive result in subjects without the specified risk. A negative likelihood ratio is the probability of a negative result in subjects without the specified risk divided by the probability of a negative result in subjects with specified risk. The term “odds ratio,” as used herein, refers to the ratio of the odds of an event occurring in one group (e.g., a healthy control group) to the odds of it occurring in another group (e.g., a cancer recurrence risk group), or to a data-based estimate of that ratio. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., a healthy control group and a cancer recurrence risk group). ROC curves are useful for plotting the performance of a particular feature (e.g., any of the biomarkers described herein and/or any item of additional biomedical information) in distinguishing or discriminating between two populations (e.g., cases having a condition and controls without the condition). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

In one embodiment, the method further comprises treating the subject. The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

As used herein the terms “effective amount” or “therapeutically effective amount” refer to an amount of an active agent as described herein that is sufficient to achieve, or contribute towards achieving, one or more desirable clinical outcomes, such as those described in the “treatment” and “prevention” descriptions above. An appropriate “effective” amount in any individual case may be determined using standard techniques known in the art, such as dose escalation studies, and may be determined taking into account such factors as the desired route of administration (e.g. systemic vs. intracranial), desired frequency of dosing, etc. Furthermore, an “effective amount” may be determined in the context of the co-administration method to be used.

Disclosed herein is a method of preventing or treating recurrence of a cancer in a subject, the method comprises a) comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference predicts the likelihood of recurrence of the cancer in the subject; and b) administering an anti-cancer agent and/or radiotherapy to the subject when the subject is found to have a likelihood of recurrence of the cancer.

Anti-cancer agents for treating NPC may include gemcitabine (Gemzar), cisplatin (Platinol), docetaxel (Taxotere), 5-fluorouracil (5-FU) or capecitabine (Xeloda). Combinations of chemotherapeutic agents can include, for example, gemcitabine (Gemzar) and cisplatin (Platinol); docetaxel (Taxotere) with cisplatin and 5-fluorouracil (5-FU); cisplatin and 5-fluorouracil; cisplatin and capecitabine (Xeloda); or docetaxel and cisplatin. Treatment of NPC may include administration of an anti-cancer agent with radiotherapy. The anti-cancer agent may also be an immunotherapy. The immunotherapy may be pembrolizumab, nivolumab or an anti-vascular endothelial growth factor therapy such as bevacizumab.

Also disclosed herein is a method of stratifying a subject into one who is suffering from a high risk or low risk cancer, the method comprises comparing the level of expression of one or more biomarkers selected from the group consisting of FGF2, C3 and GIMAP7 in a sample obtained from the subject to a reference, wherein a change in level of expression in the one or more biomarkers, or the value derived therefrom, as compared to the reference stratifies the subject into one who is suffering from a high risk or low risk cancer.

Provided herein is a kit comprising one or more nucleic acid molecules that bind specifically to an expression product of a biomarker selected from the group consisting of FGF2, C3 and GIMAP7 in a sample. The one or more nucleic acid molecules may be detectably labelled.

Provided herein is a composition comprising a sample and one or more nucleic acid molecules that bind specifically to an expression product of a biomarker selected from the group consisting of FGF2, C3 and GIMAP7.

In one embodiment, there is provided a composition or solid support comprising a plurality of DNA/mRNA complexes, wherein each DNA/mRNA complex in the plurality comprises a biomarker and a first and second DNA probe hybridized to the biomarker, wherein: the first probe is a capture probe; the second probe is a reporter probe; the biomarker is a mRNA transcript; and the plurality of DNA/mRNA complexes comprise one or more biomarkers selected from the group consisting of Fibroblast Growth Factor 2 (FGF2), Complement Component 3 (C3) and GTPase, IMAP Family Member 7 (GIMAP7).

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### Examples

Due to the intense inflammatory infiltrate in NPC tumors, gene expression signals from bulk gene expression profiling of tumors are mixed, comprising signal from tumor epithelial and inflammatory microenvironment cells. Defining gene signals unique to the distinct tumor compartments (tumor epithelial or microenvironment) remain a challenge.

Following the method of Foley et al., a microdissected gene expression profiling was performed in a discovery cohort of NPC patients recruited from Singapore. Primary NPC tumors from patients who subsequently developed recurrence or metastatic disease (Group A), and patients who did not develop recurrence or metastatic disease (Group B) were profiled. Primary, pre-treatment tumors from archival paraffin blocks of these patients were profiled. Tumor epithelial and microenvironment compartments were separately microdissected using laser-capture microdissection and library preparation performed with Smart-3SEQ. Samples were prepared in biological duplicates. Quality control was performed using tapestation and libraries quantified by qPCR. Libraries were pooled, sequenced, and demultiplexed. Raw fastq files were mapped to a concatenated human hg19 and Epstein-Barr virus genome using STAR aligner, followed by quantification of gene expression using featureCounts in the Subread package.

After filtering for high quality libraries, differential gene expression analysis was performed using the DESeq2 package in R studio, comparing the gene expression of tumors that eventually recurred and tumors that did not recur (Group A vs Group B). Seventeen gene expression libraries from 11 tumors in Group A were compared with seventeen gene expression libraries from 12 tumors in Group B.

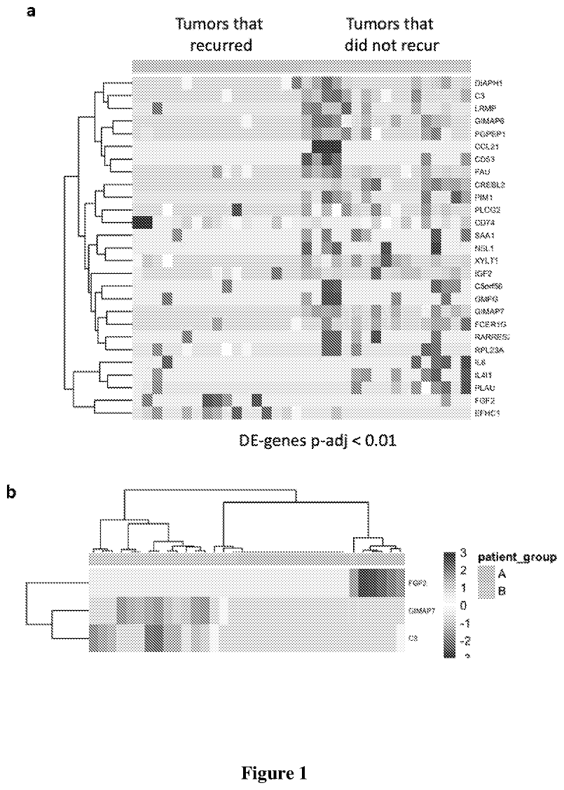

From the analysis of this cohort, twenty-seven differentially expressed genes in the tumor epithelial compartment were identified at an adjusted p-value of <0.01 (FIG. 1a). The majority of differentially expressed genes were enriched in tumors that did not recur, including several immune-related genes (C3, GIMAP genes, CCL21, IL8 etc.). Two genes were identified to be enriched in tumors which recurred (FGF2, EFHC1).

In order to refine the classifying ability of this group of genes, as well as rationalize the practical implications of using a gene expression signature to classify patients, the number of genes enriched in tumors that did not recur was reduced in an unbiased fashion using the lasso method, selecting genes at 1 (minimum) with maximum effect (|coefficient|>0.001). It was observed that C3 and GIMAP7 were optimal classifiers among the genes enriched in Group B. Based on the biological relevance of the FGF signaling pathway in cancer, FGF2 was also included in a three gene signature comprising FGF2, C3 and GIMAP7. When applied to the discovery cohort, the three gene signature accurately classified tumors into recurrent and non-recurrent groups on unsupervised clustering (FIG. 1b). A generalized linear model was developed to generate a risk score incorporating the expression of the three genes (“the three grene classifier”).

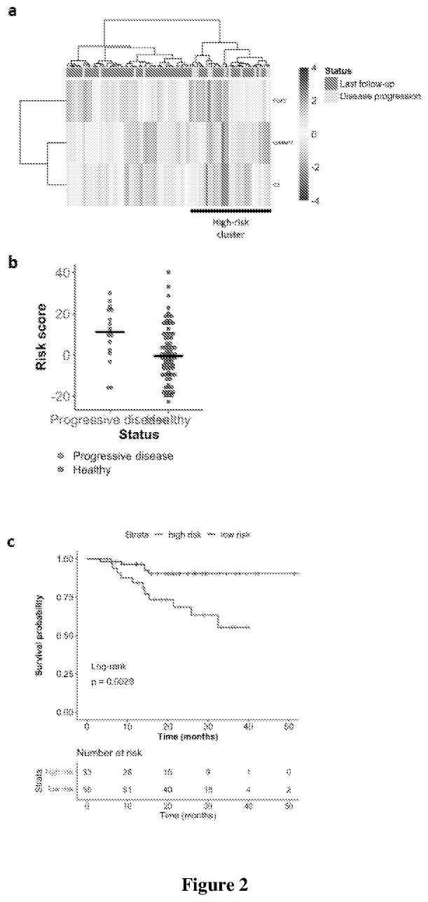

The ability of this three gene classifier to identify high-risk NPC patients in two independent cohorts was validated. The first cohort, Zhang et al.², relied on bulk RNA-Seq (not microdissected) to profile NPC tumors (n=88 tumors). The gene expression matrix and clinical outcome of this cohort is available from NCBI Gene Expression Omnibus (accession GSE102349). Despite the different method of gene expression profiling used in Lin et al., it was observed that the three gene classifier performed robustly (FIG. 2). Unsupervised hierarchical clustering showed that tumors clustered into high-risk and low-risk groups based on the expression of FGF2, C3, GIMAP7. When applying the three gene classifier, tumors in patients who developed progressive disease had higher risk scores compared to patients who remained healthy. Survival analysis based on Kaplan-Meier curve showed that patients with higher risk score tumors had poorer disease-free survival compared to patients with lower risk score tumors (p=0.0029).

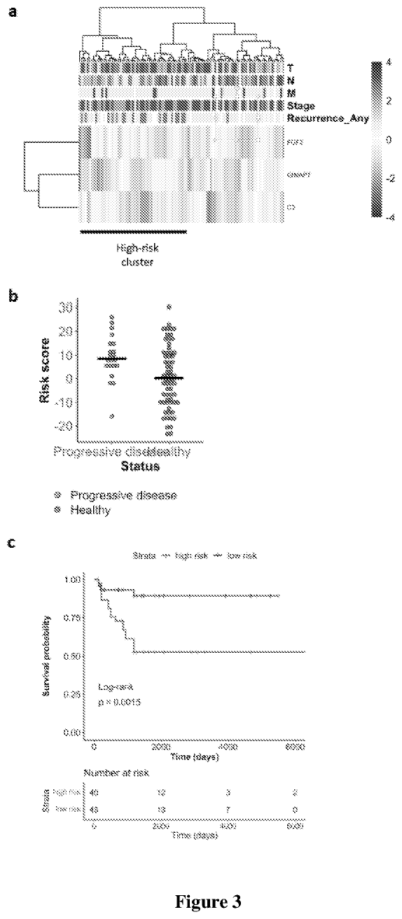

The second validation cohort is a cohort of microdissected NPC tumors from Stanford University (n=85 tumor epithelial libraries). This cohort is used for validation only and not required for the discovery of the three-gene signature. We observed that the three gene classifier performed excellently (FIG. 3). Unsupervised hierarchical clustering showed that tumors clustered spontaneously into high-risk and low-risk groups. Risk scores were also elevated in tumors of patients who developed progressive disease, compared to patients who remained healthy. Finally, survival analysis based on Kaplan-Meier curve again showed that patients with higher risk score tumors had poorer disease-free survival (p=0.0015).

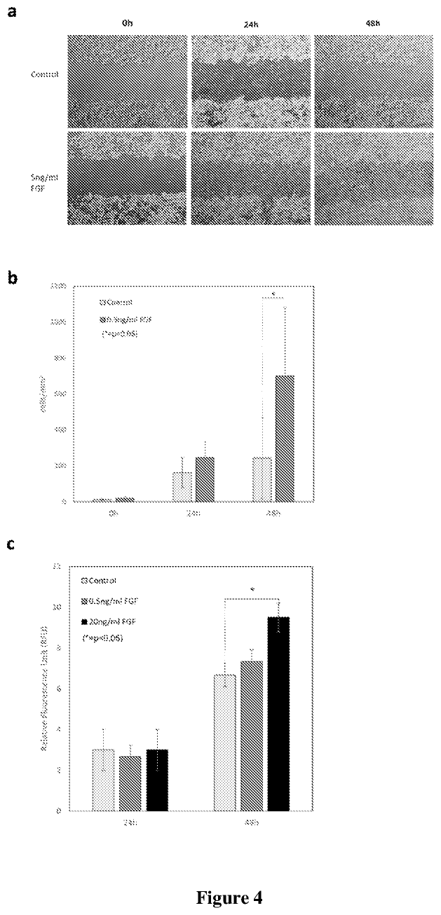

As FGF2 was observed to be upregulated in Group B tumors which developed recurrence or metastatic disease, we evaluated the ability of FGF2 to increase the cellular migration of an NPC cell line with a scratch test (cell migration assay). C666-1 cells were seeded in 6-well plate in triplicates and cultured in R10 media (RPMI 1640 supplemented with 1% non-essential amino acid (MEM Non-Essential Amino Acids Solution (100X), Gibco), 10% of fetal bovine serum (Fetal Bovine Serum, certified, United States, Gibco), and 1% of pen-strep (Penicillin-Streptomycin (10,000 U/mL), Gibco)). Upon confluency, the previous media was replaced with serum-free media. After 24 hours of serum starvation, the scratch was made using a pipette tip and the cells were washed thrice with phosphate buffered saline (PBS) to remove detached cells debris. Then the serum-free media containing 5 ng/ml FGF2 (Human FGF-basic (FGF-2/bFGF) Recombinant Protein, Gibco, dissolved in PBS) was added to the wounded cell layer. For control wells, the serum-free media containing equal volume of PBS was added. These cells were then incubated for 24 h and 48 h at 37° C. after the scratch was performed. The migration of the cells to the wounded area was monitored and captured at 0 h, 24 h, and 48 h, using Olympus IX51 Inverted Microscope coupled with DP26 Olympus digital camera. ImageJ software was done to quantify the cells that migrated. The cell migration assay was performed in biological triplicates. Statistical analysis of quantitative data was performed using the two-tailed Student's t-test, comparing FGF2-treated group to the control. The error bars on the graphs were standard deviation from obtained values from experiment. It was observed that C666-1 cells that were treated with 0.5 ng/ml of FGF2 demonstrated increased ability to migrate (FIG. 4a,b, p<0.05).

We also evaluated the ability of FGF2 to increase the invasiveness of an NPC cell line with a cell invasion assay. CytoSelect™ 96-Well Cell Invasion Assay (Basement Membrane, Fluorometric Format) kit was used according to the manufacturer's protocol. Briefly, C666-1 cells were serum-starved for 20-24 hours and then harvested. 2×10⁶ cells/ml were plated in triplicates inside the upper chamber in the serum-free media containing 5 ng/ml and 20 ng/ml FGF, with serum-free media only as control. The cells were then incubated for 24 h and 48 h at 37° C. The feeder tray contained 10% FBS as the chemoattractant for the cells. At each time point, the cells that have invaded the basement membrane and attached on the bottom of membrane were harvested using cell dissociation reagent. The cells were then incubated for 30 minutes at 37° C. Lysis buffer and fluorescent dye were then added to the detached cells and the mixture was incubated for 20 minutes at ambient temperature. The number of cells invaded the membrane was quantified by fluorescent measurements at 485 nm/528 nm using Synergy HT Microplate Reader. The cell invasion assay was performed in biological triplicates.

Statistical analysis of quantitative data was performed using the two-tailed Student's t-test, comparing FGF2-treated group to the control. One replicate from the 20 ng/ml FGF2 group was excluded due to an excessively high fluorescence reading. The error bars on the graphs were standard deviation from obtained values from experiment. It was observed that C666-1 cells treated with 20 ng/ml of FGF2 demonstrated increased invasiveness (FIG. 4c, p<0.05).

In conclusion, a gene expression classifier derived from a matched cohort of NPC tumors with and without recurrence accurately classified NPC into high-risk and low-risk groups.

### REFERENCES

Foley J W, Zhu C, Jolivet P, et al. Gene-expression profiling of single cells from archival tissue with laser-capture microdissection and Smart-3SEQ. Genome Res. 2019:doi: 10.1101/gr.234807.234118.
Zhang L, MacIsaac K D, Zhou T, et al. Genomic Analysis of Nasopharyngeal Carcinoma Reveals TME-Based Subtypes. Mol Cancer Res. 2017; 15(12):1722-1732.
Liu S L, Sun X S, Chen Q Y, et al. Development and validation of a transcriptomics-based gene signature to predict distant metastasis and guide induction chemotherapy in locoregionally advanced nasopharyngeal carcinoma. Eur J Cancer. 2022; 163:26-34.
Zhao S, Dong X, Ni X, et al. Exploration of a Novel Prognostic Risk Signature and Its Effect on the Immune Response in Nasopharyngeal Carcinoma. Front Oncol. 2021; 11:709931.
Tang X R, Li Y Q, Liang S B, et al. Development and validation of a gene expression-based signature to predict distant metastasis in locoregionally advanced nasopharyngeal carcinoma: a retrospective, multicentre, cohort study. Lancet Oncol. 2018; 19(3):382-393.

1. A method of predicting the likelihood of recurrence of a cancer in a subject, the method comprises comparing the level of expression of one or more biomarkers selected from the group consisting of Fibroblast Growth Factor 2 (FGF2), Complement Component 3 (C3) and GTPase, IMAP Family Member 7 (GIMAP7) in a sample obtained from the subject to a reference, wherein a change in level of expression in the one or more biomarkers, or a value derived therefrom, as compared to the reference predicts the likelihood of recurrence of the cancer in the subject.

2. The method of claim 1, wherein the cancer is nasopharyngeal cancer (NPC).

3. The method of claim 1 or 2, wherein the method comprises comparing the level of expression of the mRNA or protein of the one or more biomarkers.

4. The method of any one of claims 1 to 3, wherein the method comprises detecting the level of the one or more biomarkers.

5. The method of any one of claims 1 to 4, wherein the method comprises or consists of comparing the levels of FGF2, C3 and GIMAP7.

6. The method of any one of claims 1 to 5, wherein an increased, unchanged and/or decreased level of expression of FGF2, C3 and GIMAP7 as compared to a reference, predicts the likelihood of recurrence of the cancer in the subject.

7. The method of any one of claims 1 to 5, wherein the value derived therefrom is a cancer recurrence risk score obtained by processing the level of expression of one or more biomarkers selected from the group consisting of FGF2, C3 and GIMAP7 using a risk score model.

8. The method of claim 7, wherein the risk score model is generated by regression on a dataset comprising gene expression data and clinical outcome of a cohort of cancer patients.

9. The method of any one of claims 1 to 8, wherein the sample is a tissue sample.

10. A method of determining the prognosis of a cancer in a subject, the method comprises comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference indicates that the subject is likely to have a high risk cancer or a low risk cancer.

11. A method of treating cancer in a subject, the method comprises a) comparing the level of expression of one or more biomarkers selected from the group consisting of FGF2, C3 and GIMAP7 in a sample obtained from the subject to a reference, wherein a change in level of expression in the one or more biomarkers, or a value derived therefrom, as compared to the reference indicates that the subject is likely to have a high risk cancer; and b) administering an anti-cancer agent and/or radiotherapy to a subject found likely to have a high risk cancer.

12. A method of stratifying a subject into one who is suffering from a high risk or low risk cancer, the method comprises comparing the level of expression of one or more biomarkers selected from the group consisting of FGF2, C3 and GIMAP7 in a sample obtained from the subject to a reference, wherein a change in level of expression in the one or more biomarkers, or the value derived therefrom, as compared to the reference stratifies the subject into one who is suffering from a high risk or low risk cancer.

13. A composition or solid support comprising a plurality of DNA/mRNA complexes, wherein each DNA/mRNA complex in the plurality comprises a biomarker and a first and second DNA probe hybridized to the biomarker, wherein:
the first probe is a capture probe;
the second probe is a reporter probe;
the biomarker is a mRNA transcript; and
the plurality of DNA/mRNA complexes comprise one or more biomarkers selected from the group consisting of Fibroblast Growth Factor 2 (FGF2), Complement Component 3 (C3) and GTPase, IMAP Family Member 7 (GIMAP7).
